(19) United States
(12) Patent Application Publication
(10) Pub. No.: US 2003/0013635 A1
(43) Pub. Date: Jan. 16, 2003

(54) Method for the treatment of depression by inhibition of NAALADase

(76) Inventors: James L. Meyerhoff, Silver Spring, MD (US); Lucille A. Lumley, Silver Spring, MD (US)

Correspondence Address: Nash & Titus, LLC, 3415 Brookeville Road, Brookeville, MD, 20833, US

(21) Appl. No.: 10/120,712

(22) Filed: Apr. 11, 2002

Related U.S. Application Data
(60) Provisional application No. 60/282,882, filed on Apr. 11, 2001.

(51) Int. Cl. A61K 31/00

(52) U.S. Cl. 514/1

(57) ABSTRACT

A method of treating depression and depression disorders which includes administering a pharmaceutically effective amount of one or more NAALADase inhibitors to a patient. NAALADase is an enzyme that hydrolyzes the peptide NAAG, a good peptide neurotransmitter in the brain. NAAG competes with the excitatory amino acid Glu. The excitatory amino acid Glu is released in the brain upon stress and can lead to depression. Hence, the inhibition of NAALADase directly decreases levels of Glu while increasing NAAG levels, contributes to and decreases depression.

### GOVERNMENT INTEREST

The invention described herein may be manufactured, used and licensed by or for the U.S. Government.

### BACKGROUND OF THE INVENTION

1. Field of the Invention

The present invention relates to the treatment of depression by inhibiting the enzyme NAALADase in the brain. NAALADase is an enzyme that hydrolyzes the peptide NAAG, a good peptide neurotransmitter in the brain. NAAG competes with the excitatory amino acid Glu. The excitatory amino acid Glu is released in the brain upon stress and can lead to depression. Hence, the inhibition of NAALADase directly decreases levels of Glu while increasing NAAG levels, contributes toand decreases depression.

2. Brief description of Related Art

Depression affects many people in this country and abroad. Depression is a neurotic or psychotic condition marked by an inability to concentrate, insomnia, and feelings of dejection and guilt. In some cases it is mild and in other cases it can even be debilitating. Depression is now well recognized as an extremely damaging and invalidating disorder and it has a very large prevalence. It is often associated with suicidal behaviour and people afflicted have a very low quality of life. Depression is a different condition than anxiety.

Depression is also a problem in the military. The incidence of Combat Stress Reaction, which includes depression, is proportional to battle intensity and expected to range from 30% to 100% of the rate of wounding. No suitable pharmacological intervention is available, in as much as benzodiazepines interfere with leaming and memory and would therefore impair soldier performance. Benzodiazepines also are associated with dependence/withdrawal. Should this paragraph be deleted?

The use of NAALADase inhibition is a totally novel approach to treatment for depression. No other single compound elicits the same cluster of convergent physiological effects. Such a use has not been previously suggested. Other treatments are associated with undesirable profiles or unacceptable side effects. Benzodiazepines also are well-known to be associated with dependence/withdrawal. Drugs which directly block the NMDA-associated ion channel are associated with unacceptable behavioral toxicity [Tricklebank et al., 1989].

Depression and social phobias are a major medical problem in the US, accounting for much loss of workplace productivity, family stress and abuse of drugs and alcohol. Agitated depression is associated with significant risk of suicide. A new class of anxiolytic/antidepressant drug would be of immense value to medical practice, both civilian and military.

NAALDase inhibitors have been used to treat anxiety disorders but never depression. U.S. Pat. No. 6,228,888 id directed to a method of using NAALADase inhibitors for treating glutamate mediated diseases, disorders and conditions such as anxiety, anxiety disorders and memory impairment.

NAALDase inhibitors have also been implicated for the treatment of compulsive disorders, prostate diseases and cancers as recited in U.S. Pat. No. 6,348,464. However, as stated above, NAADase inhibitors have never been used to treat depression.

Other drugs have been used to treat depression. Direct approaches for reducing glutamateric tone in the central nervous system, such as blocking the NMDA-associated ion channel are associated with unacceptable behavioral toxicity [Tricklebank et al., 1989]. Is glutamatergic tone associated with depression?

Inhibition of NAALADase is a totally novel approach to decreasing glutamatergic tone in the central nervous system. Inhibition of NAALADase is a much less behaviorally disruptive way to modulate glutamatergic tone than is blockade of the ion channel, as it directly decreases Glu levels while increasing NAAG levels; NAAG in turn, indirectly reduces Glu levels by inhibiting its release via agonist effects at the presynaptic mGluR3 receptor; and NAAG is also a natural competitor with Glu at the postsynaptic NMDA receptor.

Benzodiazepines, known to be potent anxiolytics, and antipressants are these used to treat depression? If so, why are they not suitable? have sedative effects, interfere with learning and memory and would therefore impair workplace and military performance. Benzodiazepines are also well-known to be associated with dependence/withdrawal, a very serious liability.

Buspirones (e.g. Buspar) are serotonin receptor antagonists which achieve anxiolytic depression? effects, but only after chronic (i.e. 10 to 14 day) administration, a significant limitation in clinical utility. No class of pharmaceutical has consistently improved the symptoms of PTSD [Zisook et al., 2000].

Therefore, there is a need for a better treatment of depression, phyarmaceutical compositions comprising such inhibitors and method of their use.

### BRIEF DESCRIPTION OF THE FIGURES

FIG. 1 is a graph showing territoriality of socially defeated mice verses socially undefeated mice that received NAALADase inhibitor GPI 5232.

### SUMMARY OF THE INVENTION

The present invention relates to pharmaceutical compositions and method of using NAALADase inhibitors to inhibit N-Acetylated alpha-linked acidic dipeptidase (NAALADase) to indirectly reduce Glu levels by inhibiting Glu release from presynaptic neurons via full agonist effects at the presynaptic mGluR3. The invention further relates to diminishing glutamatergic tone by inhibiting NAALADase to overcome the social defeat (SD) model in the mouse and decrease depression in humans.

N-acetylaspartylglutamate (NAAG) is the most abundant peptide neurotransmitter in the brain. Glu release is increased during stress. Inhibiting glutamatergic tone by inhibiting NAALADase proves an antidepressant in the social defeat (SD) model. The SD model is a form of conditioned contextual fear, in which once-defeated mice retain a long-lasting over-generalized fear and avoidance of all mice (even non-aggressive ones) and lose normal species-appropriate territorial defense. We found that the NAALADase-inhibiting drug GPI 5232 markedly diminished this conditioned fear.

### DETAILED DESCRIPTION

There is a relationship between stress and excitatory amino acids. For example, stress stimulates release of the excitatory amino' acid, glutamate (Glu) in the locus coeruleus (LC) Singewald, N., et al., Corticotropin-releasing factor modulates basal and stress-induced excitatory amino acid release in the locus coerulus of conscious rats. Neurosci. Lett, 1996.204(1-2): p. 45-8.

The LC is a major relay station in the central nervous system response to stress, and projects to many forebrain areas, including the hippocampus. Stress has also been shown to increase extracellular Glu in the hippocampus. Lowy, M. T., L. Gault, and B. K. Yamamoto, Adrenalectomy attenuates stress-induced elevations in extracellular glutamate concentration in the hippocampus. J. Neurochem, 1993.61: p. 1957-1960.

N-acetylaspartylglutamate (NAAG) is a dipeptide endogenous to the brain and is hydrolyzed by a NAAG peptidase (NAALADase) to form the potent excitatory neurotransmitter, glutamate (Glu). Although a weak agonist at the NMDA receptor, NAAG acts as a full agonist at the presynaptic mGluR3 metabotropic glutamatergic receptor where it is equipotent with Glu and serves to decrease release of Glu into the synapse. Hence, inhibition of NAALADase increases NAAG levels, reduces Glu release via agonist effects at the mGluR3, and tends to compete with Glu at the NlMDA receptor.

NAAG indirectly reduces Glu levels by inhibiting Glu release from presynaptic neurons via full agonist effects at the presynaptic mGluR3; and NAAG also acts as a natural competitor with Glu at the postsynaptic NMDA receptor (reviewed in Coyle, J. T. The nagging question of the function of N-acetylaspartylglutamate. Neurobiology of disease. 4:231-238, 1997.; Neale, J. H., Bzdega, T. and Wroblewska, B. N-Acetylaspartylglutamate: the most abundant peptide neurotransmitter in the mammalian central nervous system. J. Neurochem. 75:443-452, 2000.

Since Glu release is increased during stress, we tested the hypothesis that diminishing glutamatergic tone by inhibiting NAALADase would prove anxiolytic and anti depressant in the social defeat (SD) model in the mouse.

SD Model

The SD model is a form of conditioned contextual fear, in which once-defeated mice retain a long-lasting over-generalized fear and avoidance of all mice (even non-aggressive ones), losing normal species-appropriate territoriality [Lumley et al., 1999] and displaying immobility in a swim test—a behavior associated with models of depression [Hebert et al., 1998].

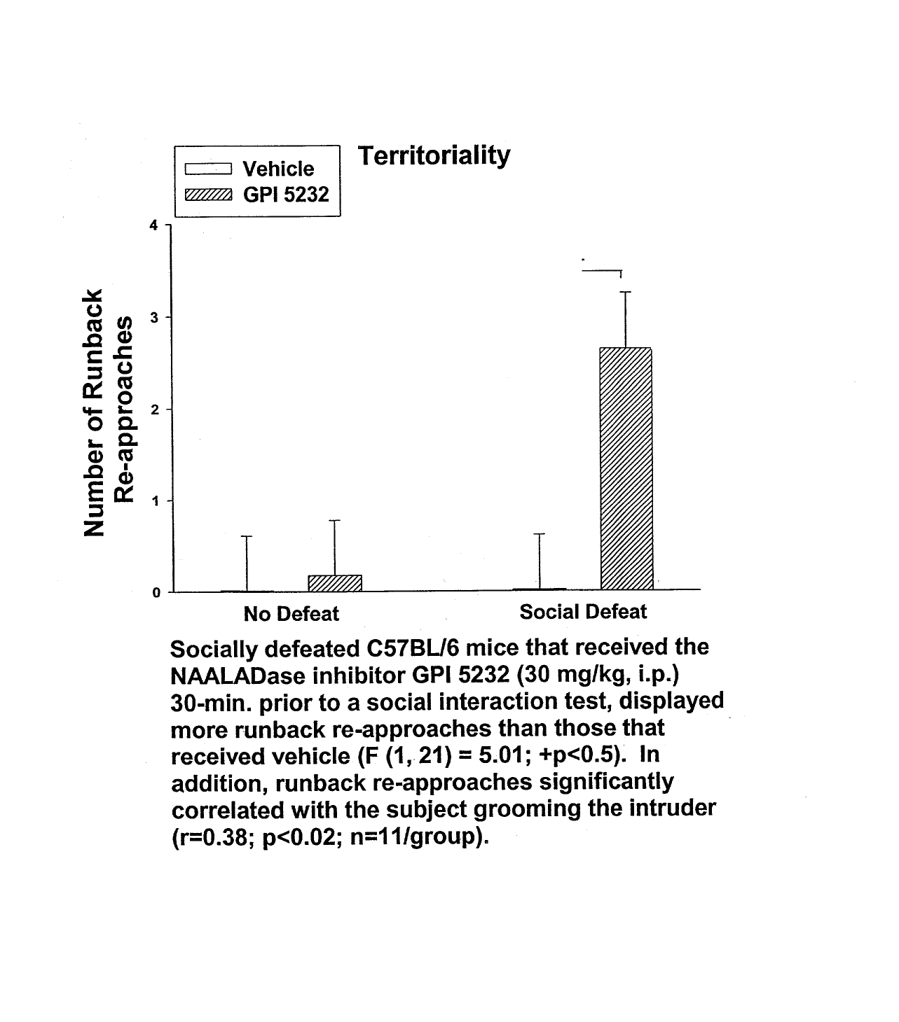

We found that acute injection of the NAALADase-inhibiting drug GPI 5232 significantly increased the display of territorial behavior in the form of runback re-approaches during social interaction with a non-aggressive intruder (see FIG. 1). Moreover, these behaviors were significantly correlated with grooming the intruder. Thus, GPI 5232 increased normal territoriality and other species-appropriate social behavior. In sum, this drug diminished behaviors associated with conditioned contextual fear. It is also believed to reduce depression.

The SD paradigm has been proposed as model for social phobia, combat stress reaction, posttraumatic stress disorder (PTSD) and depression [Hebert et al., 1998; Lumley et al., 1999]. Accordingly, the inventors use NAALADase inhibitors, NAAG analogs and NAAG potentiators in the treatment of depression disorders.

NAALADase inhibitors might also be useful for treating obsessive-compulsive disorders and eating disorders.

This invention is designed to provide relief of the symptoms of depression as well as social phobias, combat stress reaction and posttraumatic stress disorder, many of which have depression as a secondary diagnosis. Patients with these disorders frequently display avoidance behaviors and social withdrawal. The described mechanism of action is unique and without precedent for any single compound.

### EXAMPLES

### Example 1

The proposal includes testing of GPI 5000 for efficacy in four separate models: (1) neuroprotection in spinal injury; (2) agonist/antagonist activity on neuronal calcium flux; (3) anticonvulsant/neuroprotection using pilocarpine model of soman-induced neurotoxicity; (4) anxiolytic potency.

What is claimed is:

1. A method of treating depression comprising: administering to a patient a pharmaceutically effective amount of an NAALADase inhibitor.

2. The method of claim 1, wherein the NAALADase inhibitor is GPI 5232.
